(19) United States
(12) Patent Application Publication
(10) Pub. No.: US 2024/0426841 A1
(43) Pub. Date: Dec. 26, 2024

(54) KIT OF KL-6 PROTEIN ON EXOSOMES FOR AUXILIARY DIAGNOSIS OF INTERSTITIAL LUNG DISEASE AND USE THEREOF

(71) Applicants: THE FIRST AFFILIATED HOSPITAL OF GUANGZHOU MEDICAL UNIVERSITY (GUANGZHOU RESPIRATORY CENTER), Guangzhou (CN); GUANGZHOU NATIONAL LABORATORY, Guangzhou (CN); GUANGZHOU INSTITUTE OF RESPIRATORY HEALTH (GUANGZHOU INSTITUTE OF RESPIRATORY DISEASES), Guangzhou (CN)

(72) Inventors: Baoqing SUN, Guangzhou (CN); Ming DONG, Guangzhou (CN); Huimin HUANG, Guangzhou (CN); Peiyan ZHENG, Guangzhou (CN); Gaolei HU, Guangzhou (CN); Xi CHEN, Guangzhou (CN)

(21) Appl. No.: 18/826,442

(22) Filed: Sep. 6, 2024

Related U.S. Application Data
(63) Continuation of application No. PCT/CN2024/092551, filed on May 11, 2024.

(30) Foreign Application Priority Data
May 15, 2023 (CN) 202310543497.0

(51) Int. Cl. G01N 33/68

(52) U.S. Cl. CPC G01N 33/6893; G01N 2333/4725; G01N 2440/00

(57) ABSTRACT

The present application discloses a detection kit of KL-6 protein on exosomes for auxiliary diagnosis of interstitial lung diseases. A detection method includes: after pretreating the exosome sample with a sample treatment solution, exosomes containing KL-6 protein in the sample react with a coated magnetic bead working solution and a luminescent marker-labeled antibody working solution to form an immunocomplex, and a luminous intensity is detected by using a chemiluminescence method and a concentration value is calculated. The application provides, for the first time, a method for diagnosing interstitial pneumonia using a combination of KL-6 and CA125 proteins on exosomes extracted from blood, or using KL-6 alone.

### CROSS-REFERENCE TO RELATED APPLICATION

The present application is a continuation of PCT application serial No. PCT/CN2024/092551, filed on May 11, 2024, which claims the priority benefits of China patent application No. 202310543497.0, filed on May 15, 2023. The entireties of PCT application No. PCT/CN2024/092551 and China patent application No. 202310543497.0 are hereby incorporated by reference herein and made a part of this specification

### TECHNICAL FIELD

The present application relates to a technical field of protein detection, and, in particular, to a kit of KL-6 protein on exosomes for auxiliary diagnosis of an interstitial lung disease, a detection method and use thereof.

### BACKGROUND ART

Interstitial lung disease (ILD) is a generic term for clinical-pathological entities consisting of groups of different kinds of diseases with diffuse lung parenchyma, alveolar inflammation and interstitial fibrosis as a pathological primary lesions, active dyspnea, diffuse infiltration shadow on chest X-rays, restrictive ventilation disorder, reduced diffusion capacity (DLCO) and hypoxia as clinical manifestations. Early diagnosis and treatment of interstitial pneumonia can improve a prognosis of patients. Thus, in addition to means of pulmonary CT scans, lung function tests, blood gas analysis, bronchoscopy, lung biopsy and the like which are clinically used for ILD diagnosis, serological tests related to the development and prognosis of ILD have become a focus of research.

Sialylated carbohydrate antigen-6 (KL-6) is considered as a relatively promising serological marker. Current studies show that KL-6 is significantly correlated with the occurrence, progression, drug efficacy, and prognosis of ILD lesions. However, a detection KL-6 in serum still demonstrate a problem of insufficient clinical sensitivity and specificity, so that the development of diagnostic reagents with higher sensitivity and specificity for interstitial pneumonia is an important clinical demand.

Exosomes are extracellular vesicles with a diameter of 40-160 nm and an average diameter of 100 nm, which are formed by invagination of a plasma membrane and subsequent fusion with other intracellular vesicles and organelles. Various cells can secrete exosomes under both normal and pathological conditions, but research on the relationship between proteins on exosomes and interstitial pneumonia is still insufficient. KL-6 is a single-pass transmembrane protein expressed by MUC1 gene. Theoretically, membrane proteins can be retained on exosomes in the process of exosome formation and enter a circulatory system via exosomal secretion, so that the membrane proteins have significant potential clinical value for serological diagnosis. However, the detection of an expression level of the proteins on exosomes and a correlation of the proteins with interstitial pneumonia are not yet reported.

Therefore, establishing a method for judging a type and progression of interstitial pneumonia by accurately detecting lung disease-related proteins on exosomes has important clinical value and research value.

### SUMMARY

The present application aims to provide a kit for detecting KL-6 protein on exosomes and a detection method thereof, which can be used independently or in combination for auxiliary diagnosis of interstitial lung diseases.

In a first aspect, the present application provides a use of KL-6 protein on exosomes in preparing a kit for diagnosing a lung disease, which adopts the following technical solution.

Use of KL-6 protein on exosomes in preparing a kit for diagnosing a lung disease.

Further, KL-6 protein on exosomes is used as a marker of interstitial lung diseases for diagnosing interstitial pneumonia or other lung diseases.

KL-6 protein is primarily expressed on a surface of type II alveolar epithelial cells. When interstitial pneumonia occurs and a pulmonary basement membrane is damaged, type II alveolar epithelial cells proliferate to repair the damage, leading to an increase in KL-6 production, which enters into blood through a damaged basement membrane. KL-6 protein exists in blood in two forms: as free KL-6 protein and as membrane-bound KL-6 protein on extracellular vesicles (primarily exosomes). Studies have found that detecting KL-6 protein on exosomes in blood has higher clinical coincidence rate, and a detection of KL-6 protein on exosomes reduces an influence of free KL-6 protein (existing in both healthy people and diseased people) and other endogenous interferents in serum.

In a second aspect, the present application provides a use of a combination of KL-6 and CA125 on exosomes in preparing a kit for diagnosing a lung disease, which adopts the following technical solution.

Use of a combination of KL-6 and CA125 on exosomes in preparing a kit for diagnosing a lung disease.

Further, a protein combination of KL-6 and CA125 on exosomes is used as a marker of interstitial lung diseases for diagnosing interstitial pneumonia or other lung diseases.

By adopting the above technical solution, high expression of CA125 on exosomes is related to fibrosis in interstitial pneumonia or severe pneumonia. A combined detection of KL-6 and CA125 on exosomes can effectively diagnose patients in a fibrotic progression stage, thereby being beneficial to early administration of effective treatment and avoiding delay of treatment.

In a third aspect, the present application provides a kit for diagnosing a lung disease, which adopts the following technical solution.

A kit for diagnosing a lung disease, including a sample treatment solution, a magnetic bead coated with a capture antibody, a detection antibody labeled with a luminescent marker, a luminescent substrate, and a protein standard; wherein the capture antibody is a KL-6 capture antibody, and the detection antibody is a KL-6 detection antibody. In the present application, both the KL-6 capture antibody and KL-6 detection antibody are antibodies that can specifically bind to KL-6.

A kit for diagnosing a lung disease, including a sample treatment solution, a magnetic bead coated with a capture antibody, a detection antibody labeled with a luminescent marker, a luminescent substrate, and a protein standard; wherein the capture antibody is a CA125 capture antibody, and the detection antibody is a CA125 detection antibody. In the present application, both the CA125 capture antibody and CA125 detection antibody are antibodies that can specifically bind to CA125.

By adopting the above technical solutions, detecting KL-6 on exosomes independently, or detecting a combination of KL-6 on exosomes and CA125 on exosomes, can effectively diagnose patients in the fibrotic progression stage, thereby being beneficial to early administration of effective treatment and avoiding delay of treatment.

In some embodiments, the sample treatment solution is a lysis type sample treatment solution, the lysis type sample treatment solution includes the following raw materials in percentage by weight: disodium hydrogen phosphate dodecahydrate 0.5-2%, sodium dihydrogen phosphate dihydrate 0.5-2%, NaCl 0.5-3.0%, BSA 0.5-3%, Triton X-100 0.05-1%, anionic surfactant 0.1-2%, zwitterionic surfactant 0.1-5%, Tween-20 0.1-2%, EDTA-2Na 0.02-0.1%, sodium azide 0.05-0.2%, and water in balance, with a pH of 7.4.

In some embodiments, the anionic surfactant is at least one selected from a group consisting of: sodium dodecyl sulfate (SDS), sodium lauroyl sarcosinate (NLS), and lithium dodecyl sulfate (SLS).

In some embodiments, the zwitterionic surfactant is at least one selected from a group consisting of: CHAPS, sodium lauroamphoacetate, sodium cocoamphoacetate, CHAPSO, SB3-14, SB3-12, SB3-10, and SB3-8.

In some embodiments, the sample treatment solution is a non-lysis type sample treatment solution, the non-lysis type sample treatment solution includes the following raw materials in percentage by weight: disodium hydrogen phosphate dodecahydrate 0.5-2%, sodium dihydrogen phosphate dihydrate 0.5-2%, NaCl 0.5-3%, BSA 0.5-3%, zwitterionic surfactant 0.2-2%, Tween-20 0.1-2%, EDTA-2Na 0.02-0.1%, sodium azide 0.05-0.2%, and water in balance, with a pH of 7.4.

In some embodiments, the zwitterionic surfactant is at least one selected from a group consisting of: SB3-14, sodium lauroamphoacetate, sodium cocoamphoacetate, SB3-12, SB3-10, and SB3-8.

In a fourth aspect, the present application provides a method for detecting protein content on exosomes using the above kit, which adopts the following technical solution.

A method for detecting protein content on exosomes using the above kit, including the following steps: before detecting an exosome sample to be detected, performing a pretreatment on an exosome sample to be detected by mixing the exosome sample to be detected with the sample treatment solution to obtain a pretreated exosome sample to be detected.

After pretreating the exosome sample with the sample treatment solution, exosomes containing KL-6 (or CA125) protein in the sample react with a coated magnetic bead working solution and a luminescent marker-labeled antibody working solution to form an immunocomplex, and a luminous intensity is detected by using a chemiluminescence method and a concentration value is calculated.

In a specific embodiment, a method for detecting KL-6 (or CA125) protein content on exosomes, including the following steps:
(1) Performing a pretreatment on an exosome sample to be detected: Mixing the exosome sample to be detected with the sample treatment solution for reaction;
(2) Mixing a pretreated exosome sample to be detected obtained in step (1) with a coated magnetic bead working solution for reaction to obtain a magnetic bead-exosome/antigen complex;
(3) Mixing the magnetic bead-exosome/antigen complex obtained in step (2) with a luminescent marker-labeled antibody working solution for reaction to obtain a magnetic bead-exosome/antigen-detection antibody complex;
(4) Mixing the magnetic bead-exosome/antigen-detection antibody complex obtained in step (3) with a luminescent substrate for reaction, and detecting a luminous intensity;
(5) Calculating the protein content on the exosome sample to be detected based on the luminous intensity detected in step (4) by using a standard curve method.

In a specific embodiment, a volume ratio of the exosome sample to be detected to the sample treatment solution in step (1) is 1: (2-30).

For example, the volume ratio of the exosome sample to be detected to the sample treatment solution in step (1) may be 1:2, 1:5, 1:10, 1:12, 1:15, 1:18, 1:19, 1:20, 1:22, 1:25, 1:28, or 1:30.

In a specific embodiment, a reaction time of the pretreatment in step (1) is 1-120 min; preferably, the reaction time is 1-10 min.

For example, the reaction time may be 1 min, 2 min, 3 min, 4 min, 5 min, 6 min, 7 min, 8 min, 9 min or 10 min, etc.

In a specific embodiment, a reaction temperature of the pretreatment in step (1) is 25-42° C.; preferably, the reaction temperature is 35-40° C.

For example, the reaction temperature may be 35° C., 36° C., 37° C., 38° C., 39° C., or 40° C., etc.

In a specific embodiment, a reaction time in step (2) and step (3) is 1-120 min; preferably, the reaction time is 5-15 min.

For example, the reaction time may be 5 min, 6 min, 7 min, 8 min, 9 min, 10 min, 11 min, 12 min, 13 min, 14 min, or 15 min.

In a specific embodiment, a reaction temperature in step (2) and step (3) is 25-42° C.; preferably, the reaction temperature is 35-40° C.

For example, the reaction temperature may be 35° C., 36° C., 37° C., 38° C., 39° C., or 40° C.

In the above steps, when using a non-lysis sample treatment solution, and a treated sample reacts with a coated magnetic bead to form a magnetic bead-exosome KL-6 antigen complex; when using a lysing sample treatment solution, the treated sample reacts with the magnetic bead coating to form a magnetic bead-KL-6 antigen complex.

In the present application, the lysis sample treatment solution can damage a membrane structure of the exosome, and the non-lysis sample treatment solution cannot damage a membrane structure of the exosome. When using the non-lysis sample treatment solution for pretreating the sample, concurrent incubation of a mixed sample is required. When using the lysis sample treatment solution for pretreating the exosome sample, a next step can be proceeded without incubation.

The exosome sample to be detected in the present application can be prepared from any one of the following test samples: whole blood, serum, plasma, bronchoalveolar perfusate or urine.

A preparation method of the exosome sample to be detected in the present application is any one of a full-automatic exosome extraction instrument or a manual extraction method (such as a centrifugation method, a kit method, an ultrafiltration method and a magnetic bead immunization method); and preferably using the full-automatic exosome extraction instrument or the kit method.

In some common exosome extraction methods (such as a kit method, a magnetic bead immunization method, and a specific fully automatic instrument extraction method), an eluent is often required to elute the exosome from an adsorption carrier. These eluents are mainly solutions containing high concentrations of salt ions or surfactants, which can cause dehydration of exosomes and lead to aggregation, and may also alter a conformation of proteins on exosomes. These factors can lead to poor accuracy and reproducibility of detection results of protein on exosomes, making the detection results unsuitable for clinical diagnosis. Research has found that diluting the exosome sample with a sample dilution solution can effectively improve a signal-to-noise ratio of detection, but requires a longer reaction time and is not suitable for automated detection. Based on this, by coordinating with a sample treatment solution with a specific concentration range, the reaction time can be further reduced, a detection efficiency is effectively improved, the signal-to-noise ratio of detection is improved, and the full-automatic detection of protein on exosome is realized.

By pretreating the exosome sample with the sample treatment solution, a detection accuracy can be further improved, and a detection time can be greatly reduced without additional incubation time.

In a specific embodiment, a preparation method of a capture antibody-coated magnetic bead (also referred to as a coated magnetic bead) is as follows.

Magnetic bead activation: adding 5-20 mg of magnetic beads (magnetic bead concentration of 5-20 mg/mL) to 1 mL of magnetic bead activation buffer (aqueous solution containing MES at a mass concentration of 1%), then adding 25 μL of EDC solution with a concentration of 20 mg/mL (final concentration 0.5 mg/mL) and 25 μL of NHS solution with a concentration of 40 mg/mL (final concentration of 1.0 mg/mL); reacting at room temperature for 15-120 min to activate the magnetic beads, then removing a supernatant by magnetic separation.

Antibody coupling: resuspending the activated magnetic beads in 1 mL of magnetic bead coupling buffer (aqueous solution containing MES at a mass concentration of 1%) at a concentration of 5-20 mg/mL; adding 100-300 μg of KL-6 capture antibody to be coated (final concentration of about 100-300 μg/mL), incubating at room temperature for 1-2 hours to obtain a solution containing a magnetic bead-antibody conjugate.

Blocking: adding 100 μL of blocker (aqueous solution containing bovine serum albumin at a mass concentration of 1%) to the solution containing the magnetic bead-antibody conjugate, a volume ratio of the solution containing the magnetic bead-antibody conjugate to the blocker is 1:0.1, and incubating at room temperature for 1-4 hours. After removing a supernatant by magnetic separation, adding 1 mL of magnetic bead dilution solution to obtain the capture antibody-coated magnetic bead; wherein, the bead dilution solution is a PBS buffer with 1% w/v BSA and 0.1% w/v proclin 300.

Preparation of coated magnetic bead working solution: Diluting the capture antibody-coated magnetic bead to a target concentration using a coated magnetic bead dilution solution, a concentration of the capture antibody-coated magnetic bead after dilution is in the range of 0.1 to 1.5 mg/mL; particularly, a preferred concentration is in the range of 0.3 to 0.8 mg/mL.

The coated magnetic bead dilution solution includes the following components: TRIS 0.05-0.1%, NaCl 0.9-3.0%, BSA 1-3%, Tween-20 0.1-0.5%, sodium azide 0.1%, and water in balance, with a pH of 7.5.

A preparation method of a luminescent marker-labeled detection antibody (also referred to as a luminescent marker) is as follows.

Preparation of acridinium ester stock solution: dissolving acridinium ester in anhydrous DMSO to prepare an acridinium ester stock solution (final concentration of 2.5 mg/mL).

Preparation of acridinium ester working solution: taking 10 μL of the acridinium ester stock solution (final concentration of 2.5 mg/mL), diluting 10 times with 90 μL of anhydrous DMSO to prepare an acridinium ester working solution (final concentration of 0.25 mg/mL).

Labeling reaction: diluting 200-400 μg of KL-6 detection antibody to 300 μL (antibody final concentration of 0.66-1.33 mg/mL) using 0.2M NaHCO₃ solution (pH=9.0), adding 10 μL of the acridinium ester working solution (antibody final concentration of about 0.008 mg/mL), wrapping with tin foil, and labeling at room temperature for 0.5-2 h.

Quenching reaction: adding 100 μL of labeling termination buffer, mixing at room temperature for 30-120 min; wherein, the buffer is a 0.2M NaHCO₃ solution containing 10% w/v lysine, with a pH of 9.0.

Purification: purifying a labeled antibody using a desalting column, collecting an acridine-labeled protein fraction and adjusting concentration to obtain the luminescent marker-labeled detection antibody.

Preparation of luminescent marker-labeled antibody working solution (also referred to as luminescent marker labeled working solution): diluting the luminescent marker-labeled detection antibody to a target concentration using a luminescent marker labeled dilution solution, a concentration after dilution is in the range of 0.05 to 1.00 μg/mL, and particularly, a preferred concentration is in the range of 0.1-0.6 μg/mL.

The luminescent marker labeled dilution solution includes the following components: MES 0.05-0.1%, NaCl 0.9-3.0%, BSA 1-3%, Tween-20 0.1-0.5%, sodium azide 0.1%, and water in balance, with a pH of 6.5.

The luminescent marker used in the preparation of the luminescent marker-labeled detection antibody is any one selected from a group consisting of: acridinium ester, alkaline phosphatase, and horseradish peroxidase.

In summary, the present application includes at least one of the following beneficial technical effects.

The present application provides a method for auxiliary diagnosis of interstitial lung diseases using KL-6 protein on exosomes, which reduces an influence of free KL-6 protein and other interferents in serum compared with a detection of KL-6 in serum, has higher specificity and sensitivity compared with a traditional detection kit of KL-6 in serum, and has good popularization prospect.

The present application provides a non-lysis type sample pretreating method and a lysis type sample pretreating method, reduces an influence of components in eluent on a detection of protein on exosomes, effectively reduces a detection time, and is beneficial to a realization of a full-automatic detection of protein on exosomes.

For the first time, the present application provides a method using a combination of KL-6 and CA125 protein on exosomes for auxiliary diagnosis of interstitial lung diseases, which has important clinical value in auxiliary diagnosis of interstitial lung diseases.

By establishing a method for detecting KL-6 protein on exosomes, the present application realizes a stable detection of protein on exosomes, which is of significant value for a study of protein on exosomes.

### BRIEF DESCRIPTION OF THE DRAWINGS

FIG. 1 is a bar chart comparing a detection result of healthy people samples and ILD patient samples in Test Example 2.

FIG. 2 is a ROC curve of KL-6 in serum and KL-6 on exosomes in Test Example 3.

### DETAILED DESCRIPTION

For better understanding of the present application, the present application will be further described in detail below with reference to the following Examples. These Examples are illustrative and do not limit the scope of the application. Various simple modifications to technical solutions of the present application, within a technical conception of the present application, are considered within a scope of protection of the present application. Unless specifically stated otherwise, all raw materials or components used in the present application can be obtained by commercial means or conventional methods.

Definitions of abbreviations and key terms used in Examples

| | |
|---|---|
| BSA | Bovine Serum Albumin |
| Tween-20 | Polysorbate 20 |
| EDTA-2Na | Ethylenediaminetetraacetic acid disodium salt |
| MES | 2-(N-morpholino)ethanesulfonic acid |
| EDC | 1-Ethyl-3-(3-dimethylaminopropyl) carbodiimide |
| | hydrochloride |
| NHS | N-Hydroxysuccinimide |
| Triton X-100 | Polyethylene glycol octylphenyl ether |
| CHAPS | 3-[(3-Cholamidopropyl)dimethylammonio]-1- |
| | propanesulfonate |
| SB3-14 | 3-(N,N-Dimethylmyristylammonio)propanesulfonate |

### Example 1

A method for detecting KL-6 protein on exosomes included the following steps.

### (1) Preparation of Coated Magnetic Bead.

Magnetic bead activation: 10 mg of magnetic beads (magnetic bead concentration of 10 mg/mL) was added to 1 mL of magnetic bead activation buffer (aqueous solution containing MES at a mass concentration of 1%). Then 25 μL of EDC solution with a concentration of 20 mg/mL (final concentration 0.5 mg/mL) and 25 μL of NHS solution with a concentration of 40 mg/ml (final concentration of 1.0 mg/mL) were added to obtain a mixture. The mixture was reacted at room temperature for 30 min to activate the magnetic beads, then removed of supernatant by magnetic separation.

Antibody coupling: the activated magnetic beads was resuspend in 1 mL of magnetic bead coupling buffer (aqueous solution containing MES at a mass concentration of 1%) at a concentration of 10 mg/mL. 200 μg of the KL-6 capture antibody (final concentration of about 200 μg/mL) was added and incubated at room temperature for 2 hours to obtain a solution containing a magnetic bead-antibody conjugate.

Blocking: 100 μL of blocker (containing a 1% w/v bovine serum albumin solution) was added to the solution containing the magnetic bead-antibody conjugate, a volume ratio of solution containing the magnetic bead-antibody conjugate to blocker was 1:0.1, and a result solution was incubated at room temperature for 2 hours. After removed a supernatant by magnetic separation, 1 mL of magnetic bead dilution solution was added to obtain a capture antibody-coated magnetic bead (a KL-6 coated magnetic bead); wherein, the magnetic bead dilution solution was a PBS buffer with 1% w/v BSA and 0.1% w/v proclin 300.

Preparation of coated magnetic bead working solution: the KL-6 coated magnetic bead was diluted using a coated magnetic bead dilution solution to obtain the coated magnetic bead working solution with a concentration of 0.5 mg/mL; and the coated magnetic bead dilution solution included the following components: TRIS 0.1%, NaCl 2%, BSA 3%, Tween-20 0.5%, sodium azide 0.1%, and water in balance, with a pH of 7.5.

### (2) Preparation of Luminescent Marker-Detection Antibody Complex.

Preparation of acridinium ester stock solution: acridinium ester was dissolved in anhydrous DMSO to prepare an acridinium ester stock solution (final concentration of 2.5 mg/mL).

Preparation of acridinium ester working solution: 10 μL of the acridinium ester stock solution (final concentration of 2.5 mg/mL) was taken, then diluted 10 times with 90 μL of anhydrous DMSO to prepare an acridinium ester working solution (final concentration of 0.25 mg/mL).

Labeling reaction: 300 μg of KL-6 detection antibody was diluted to 300 μL (antibody final concentration of 1 mg/mL) using 0.2M NaHCO₃ solution (pH-9.0); 10 μL of the acridinium ester working solution was added (antibody final concentration of about 0.008 mg/mL), then a result solution was wrapped with tin foil and labeled at room temperature for 0.5 hours to botain a labeled mixture.

Quenching reaction: 100 μL of labeling termination buffer was added to the labeled mixture, then mixed at room temperature for 30 minutes; wherein, the buffer was a 0.2M NaHCO₃ solution containing 10% w/v lysine, with a pH of 9.0.

Purification: a labeled antibody was purified using a desalting column, an acridine-labeled protein fraction was collected, and a concentration of which was adjusted to obtain a luminescent marker-labeled detection antibody (KL-6 detection antibody labeled with acridinium ester).

Preparation of luminescent marker-labeled antibody working solution (also referred to as acridinium ester labeled working solution): the luminescent marker-labeled detection antibody was diluted using an acridine este labeled dilution solution to obtain the acridine ester labeled working solution with a concentration of 0.4 μg/mL; wherein, the acridine este labeled dilution solution included the following components: MES 0.1%, NaCl 2.0%, BSA 3%, Tween-20 0.1%, sodium azide 0.1%, and water in balance, with a pH of 6.5.

### (3) Pretreatment of Exosome Sample.

After extracting exosomes using a kit method, performed a pretreatment on the exosome sample: 10 μL of the exosome sample was mixed with 190 μL of sample treatment solution, then incubated at 37° C. for 5 min.

In particular, the sample treatment solution was a non-lysis type sample treatment solution and the non-lysis type sample treatment solution included the following raw materials in percentage by weight: disodium hydrogen phosphate dodecahydrate 0.6%, sodium dihydrogen phosphate dihydrate 0.6%, NaCl 0.9%, BSA 1%, SB3-14 0.4%, Tween-20 0.1%, EDTA-2Na 0.05%, sodium azide 0.1%, and water in balance, with a pH of 7.4.

### (4) Detection and Calculation of KL-6 Content on Exosome Sample to be Detected.

20 μL of the pretreated sample was taken and added 50 μL of the coated magnetic bead working solution, a result solution was mixed and incubated at 37° C. for 5 min. After magnetic separation, a precipitate in the result solution was washed to remove unbound materials, then a supernatant was removed to obtain a magnetic bead-exosome complex.

50 μL of the luminescent marker-labeled antibody working solution (acridinium ester labeled working solution) was added to a reaction cup containing the magnetic bead-exosome complex, a result solution was mixed and incubated at 37° C. for 5 min. After magnetic separation, a precipitate in the result solution was washed to remove unbound materials, then a supernatant was removed to obtain a magnetic bead-exosome-detection antibody complex.

100 μL of AE pre-excitation solution and 100 μL of excitation solution were added to a reaction cup containing the magnetic bead-exosome-detection antibody complex, a result solution was mixed, then a maximum luminescence intensity was measured. A standard curve was fitted based on the luminescence intensity of standard samples, and the KL-6 content on the sample to be detected was calculated using the standard curve.

### Example 2

The main difference from Example 1 is that: the sample treatment solution used was different.

The sample treatment solution was a non-lysis type sample treatment solution and the non-lysis type sample treatment solution included the following raw materials in percentage by weight: disodium hydrogen phosphate dodecahydrate 1.0%, sodium dihydrogen phosphate dihydrate 1.0%, NaCl 0.5%, BSA 2%, sodium lauroamphoacetate 0.5%, Tween-20 0.2%, EDTA-2Na 0.05%, sodium azide 0.1%, and water in balance, with a pH of 7.4.

### Example 3

The main difference from Example 1 is that: the sample treatment solution used was different.

The sample treatment solution was a non-lysis type sample treatment solution and the non-lysis type sample treatment solution included the following raw materials in percentage by weight: disodium hydrogen phosphate dodecahydrate 0.5%, sodium dihydrogen phosphate dihydrate 0.5%, NaCl 0.5%, BSA 0.5%, sodium cocoamphoacetate 0.2%, Tween-20 0.5%, EDTA-2Na 0.02%, sodium azide 0.05%, and water in balance, with a pH of 7.4.

### Example 4

The main difference from Example 1 is that: the sample treatment solution used was different.

The sample treatment solution was a non-lysis type sample treatment solution and the non-lysis type sample treatment solution included the following raw materials in percentage by weight: disodium hydrogen phosphate dodecahydrate 2%, sodium dihydrogen phosphate dihydrate 2%, NaCl 0.8%, BSA 3%, sodium cocoamphoacetate 1%, Tween-20 0.5%, EDTA-2Na 0.1%, sodium azide 0.2%, and water in balance, with a pH of 7.4.

### Example 5

A method for detecting KL-6 protein on exosomes included the following steps.

### (1) Preparation of Coated Magnetic Bead.

Magnetic bead activation: 10 mg of magnetic beads (magnetic bead concentration of 10 mg/mL) was added to 1 mL of magnetic bead activation buffer (aqueous solution containing MES at a mass concentration of 1%). Then 25 μL of EDC solution with a concentration of 20 mg/ml (final concentration 0.5 mg/mL) and 25 μL of NHS solution with a concentration of 40 mg/ml (final concentration of 1.0 mg/mL) were added to obtain a mixture. The mixture was reacted at room temperature for 30 min to activate the magnetic beads, then removed a supernatant by magnetic separation.

Antibody coupling: the activated magnetic beads was resuspend in 1 mL of magnetic bead coupling buffer (aqueous solution containing MES at a mass concentration of 1%) at a concentration of 10 mg/mL. 200 μg of the KL-6 capture antibody (final concentration of about 200 μg/mL) was added and incubated at room temperature for 2 hours to obtain a solution containing a magnetic bead-antibody conjugate.

Blocking: 100 μL of blocker (containing a 1% w/v bovine serum albumin solution) was added to the solution containing the magnetic bead-antibody conjugate, a volume ratio of solution containing the magnetic bead-antibody conjugate to blocker was 1:0.1, and a result solution was incubated at room temperature for 2 hours. After removed a supernatant by magnetic separation, 1 mL of magnetic bead dilution solution was added to obtain a capture antibody-coated magnetic bead (a KL-6 coated magnetic bead); wherein, the magnetic bead dilution solution was a PBS buffer with 1% w/v BSA and 0.1% w/v proclin 300.

Preparation of coated magnetic bead working solution: the KL-6 coated magnetic bead was diluted using a coated magnetic bead dilution solution to obtain the coated magnetic bead working solution with a concentration of 0.5 mg/mL; the coated magnetic bead dilution solution included the following components: TRIS 0.1%, NaCl 2%, BSA 3%, Tween-20 0.5%, sodium azide 0.1%, and water in balance, with a pH of 7.5.

### (2) Preparation of Luminescent Marker-Detection Antibody Complex.

Preparation of acridinium ester stock solution: acridinium ester was dissolved in anhydrous DMSO to prepare an acridinium ester stock solution (final concentration of 2.5 mg/mL).

Preparation of acridinium ester working solution: 10 μL of the acridinium ester stock solution (final concentration of 2.5 mg/mL) was taken, then diluted 10 times with 90 μL of anhydrous DMSO to prepare an acridinium ester working solution (final concentration of 0.25 mg/mL).

Labeling reaction: 300 μg of KL-6 detection antibody was diluted to 300 μL (antibody final concentration of 1 mg/mL) using 0.2M NaHCO₃ solution (pH=9.0); 10 μL of the acridinium ester working solution was added (antibody final concentration of about 0.008 mg/mL), then a result solution was wrapped with tin foil and labeled at room temperature for 0.5 hours to botain a labeled mixture.

Quenching reaction: 100 μL of labeling termination buffer was added to the labeled mixture, then mixed at room temperature for 30 minutes; wherein, the buffer was a 0.2M NaHCO₃ solution containing 10% w/v lysine, with a pH of 9.0.

Purification: a labeled antibody was purified using a desalting column, an acridine-labeled protein fraction was collected, and a concentration of which was adjusted to obtain a luminescent marker-labeled detection antibody (KL-6 detection antibody labeled with acridinium ester).

Preparation of luminescent marker-labeled antibody working solution (also referred to as acridinium ester labeled working solution): the luminescent marker-labeled detection antibody was diluted using an acridine este labeled dilution solution to obtain the acridine este labeled working solution with a concentration of 0.4 μg/mL; wherein, the acridine este labeled dilution solution included the following components: MES 0.1%, NaCl 2.0%, BSA 3%, Tween-20 0.1%, sodium azide 0.1%, and water in balance, with a pH of 6.5.

### (3) Pretreatment of Exosome Sample.

After extracting exosomes using a kit method, performed a pretreatment on the exosome sample: 10 μL of the exosome sample was mixed with 190 μL of sample treatment solution, a result mixture was reacted at 37° C. for 5 min, and vortexed to mix thoroughly.

In particular, the sample treatment solution was a lysis type sample treatment solution and the lysis type sample treatment solution included the following raw materials in percentage by weight: disodium hydrogen phosphate dodecahydrate 0.6%, sodium dihydrogen phosphate dihydrate 0.6%, NaCl 0.9%, BSA 1%, Triton X-100 1%, sodium dodecyl sulfate SDS 0.2%, CHAPS 1%, Tween-20 0.1%, EDTA-2Na 0.05%, sodium azide 0.1%, and water in balance, with a pH of 7.4.

### (4) Detection and Calculation of KL-6 Content on Exosome Sample to be Detected.

20 μL of the pretreated sample was taken and added 50 μL of the coated magnetic bead working solution, a result solution was mixed and reacted at 37° C. for 5 min. After magnetic separation, a precipitate in the result solution was washed to remove unbound materials, then a supernatant was removed to obtain a magnetic bead-antigen complex.

50 μL of the luminescent marker-labeled antibody working solution (acridinium ester labeled working solution) was added to a reaction cup containing the magnetic bead-antigen complex, a result solution was mixed and reacted at 37° C. for 5 min. After magnetic separation, a precipitate in the result solution was washed to remove unbound materials, then a supernatant was removed to obtain a magnetic bead-antigen-detection antibody complex.

100 μL of AE pre-excitation solution and 100 μL of excitation solution were added to a reaction cup containing the magnetic bead-antigen-detection antibody complex, a result solution was mixed, then a maximum luminescence intensity was measured. A standard curve was fitted based on the luminescence intensity of standard samples, and the KL-6 content on the sample to be detected was calculated using the standard curve.

### Example 6

The main difference from Example 5 is that: the sample treatment solution used was different.

The sample treatment solution was a lysis type sample treatment solution and the lysis type sample treatment solution included the following raw materials in percentage by weight: disodium hydrogen phosphate dodecahydrate 0.5%, sodium dihydrogen phosphate dihydrate 0.5%, NaCl 0.5%, BSA 2%, Triton X-100 0.5%, sodium dodecyl sulfate SDS 0.1%, SB3-8 0.5%, Tween-20 0.2%, EDTA-2Na 0.05%, sodium azide 0.1%, and water in balance, with a pH of 7.4.

### Example 7

The main difference from Example 5 is that: the sample treatment solution used was different.

The sample treatment solution was a lysis type sample treatment solution and the lysis type sample treatment solution included the following raw materials in percentage by weight: disodium hydrogen phosphate dodecahydrate 0.5%, sodium dihydrogen phosphate dihydrate 0.5%, NaCl 0.5%, BSA 0.5%, Triton X-100 0.05%, sodium dodecyl sarcosinate (NLS) 0.2%, sodium lauroamphoacetate 0.5%, Tween-20 0.1%, EDTA-2Na 0.02%, sodium azide 0.05%, and water in balance, with a pH of 7.4.

### Example 8

The main difference from Example 5 is that: the sample treatment solution used was different.

The sample treatment solution was a lysis type sample treatment solution and the lysis type sample treatment solution included the following raw materials in percentage by weight: disodium hydrogen phosphate dodecahydrate 1%, sodium dihydrogen phosphate dihydrate 1%, NaCl 0.9%, BSA 3%, Triton X-100 1%, sodium dodecyl sarcosinate (NLS) 0.2%, CHAPS 1%, Tween-20 2%, EDTA-2Na 0.1%, sodium azide 0.2%, and water in balance, with a pH of 7.4.

### Example 9

The main difference from Example 1 is that: the capture antibody and detection antibody used were different.

In this example, the capture antibody used was a CA125 capture antibody, and the detection antibody used was a CA125 detection antibody.

### Comparative Example

### Comparative Example 1

The difference between this Comparative Example and Example 1 is that: the sample was not pretreated.

### Comparative Example 2

The difference between this Comparative Example and Example 1 is that: the sample was pretreated with PBS buffer.

In particular, the PBS buffer included the following raw materials in percentage by weight: NaCl 0.8%, KCl 0.02%, potassium dihydrogen phosphate 0.144%, dipotassium hydrogen phosphate 0.024%, and water in balance, with a pH of 7.4.

### Comparative Example 3

The difference between this Comparative Example and Example 1 is that: the exosome sample was pretreated with a commercially available sample diluent.

A manufacturer of the sample diluent is Amyjet Scientific Co., ltd, product code: ICT-647.

### Comparative Example 4

The difference between this Comparative Example and Example 5 is that: the sample was not pretreated.

### Comparative Example 5

The difference between this Comparative Example and Example 5 is that: the sample was pretreated with PBS buffer.

In particular, the PBS buffer included the following raw materials in percentage by weight: NaCl 0.8%, KCl 0.02%, potassium dihydrogen phosphate 0.144%, dipotassium hydrogen phosphate 0.024%, and water in balance, with a pH of 7.4.

### Comparative Example 6

The difference between this Comparative Example and Example 5 is that: the exosome sample was pretreated with a commercially available sample diluent.

A manufacturer of the sample diluent is Amyjet Scientific Co., ltd, product code: ICT-647.

### Test Example

### Test Example 1: Influence of Different Pretreatment Methods on Detection Results of KL-6 Protein on Exosomes

After extracting exosomes from KL-6 positive serum samples using a kit, the exosomes were diluted using PBS buffer solution for 2 times and 4 times, then a content of KL-6 protein on exosomes was detected on a full-automatic chemiluminescence immunoassay analyzer (Model: Shine i2910) by using methods provided in Examples 1-8 and Comparative Examples 1-6, respectively. Dilution recovery rates were calculated based on luminescence values of undiluted exosomes, 2-time diluted exosomes and 4-time diluted exosomes. After dilution, the diluted exosomes were left at room temperature for more than 1 hour and then tested simultaneously with the undiluted exosomes.

TABLE 1
Influence of different preprocessing methods on
detection results of KL-6 protein on exosomes

| | | 2-time diluted exosomes | | 4-time diluted exosomes | |
|---|---|---|---|---|---|
| | Undiluted | | Dilution | | Dilution |
| | exosomes | | recovery rate | | recovery rate |
| | Average | Average | (undiluted | Average | (undiluted |
| | luminescence | luminescence | exosomes/ | luminescence | exosomes/ |
| | value | value | 2-time diluted | value | 4-time diluted |
| Groups | (RLU) | (RLU) | exosomes) | (RLU) | exosomes) |
| Example 1 | 565690 | 316009 | 112% | 168122 | 119% |
| Example 2 | 552215 | 324091 | 117% | 165882 | 120% |
| Example 3 | 576076 | 299368 | 104% | 157353 | 109% |
| Example 4 | 506076 | 279368 | 110% | 147861 | 117% |
| Example 5 | 447557 | 233779 | 104% | 118058 | 106% |
| Example 6 | 399776 | 198169 | 99% | 99683 | 100% |
| Example 7 | 357557 | 193779 | 108% | 98058 | 110% |
| Example 8 | 419754 | 205471 | 98% | 110654 | 105% |
| Comparative | 504048 | 521502 | 207% | 296370 | 235% |
| Example 1 | | | | | |
| Comparative | 485183 | 288772 | 119% | 151793 | 125% |
| Example 2 | | | | | |
| Comparative | 544176 | 335267 | 123% | 177125 | 130% |
| Example 3 | | | | | |
| Comparative | 485147 | 495841 | 204% | 258411 | 213% |
| Example 4 | | | | | |
| Comparative | 498517 | 359784 | 144% | 189854 | 152% |
| Example 5 | | | | | |
| Comparative | 452895 | 295410 | 130% | 165452 | 146% |
| Example 6 | | | | | |

Referring to Examples 1-4, Comparative Examples 1-3 and Table 1, the dilution recovery rate of a reagent detection result by using pretreatment methods of Examples 1˜4 to detect protein on exosomes is significantly better than that of Comparative Examples 1-3. This indicates that the dilution recovery rate of the reagent detection result by using the pretreatment method of the present application to detect protein on exosomes is significantly better than methods without pretreatment or using a conventional diluent for pretreatment, or using the commercially available sample diluent for pretreatment. In the present application, by using the non-lysis sample treatment solution with a specific concentration range, an incubation time can be further reduced, a detection efficiency and signal-to-noise ratio can be effectively improved, and a full-automatic detection of protein on exosomes can be achieved.

Referring to Examples 5-8, Comparative Examples 4-6 and Table 1, the dilution recovery rate of a reagent detection result by using pretreatment methods of Examples 5-8 to detect protein on exosomes is significantly better than that of Comparative Examples 4-6. This indicates that the dilution recovery rate of the reagent detection result by using the pretreatment method of the present application to detect protein on exosomes is significantly better than methods without pretreatment or using a conventional diluent for pretreatment, or using the commercially available sample diluent for pretreatment. In the present application, by using the lysis sample treatment solution with a specific concentration range, an incubation time can be further reduced, a detection efficiency and signal-to-noise ratio can be effectively improved, and a full-automatic detection of protein on exosomes can be achieved.

When the non-lysis sample treatment solution was used for pretreating the sample, concurrent incubation with a mixed sample is required. When the lysis sample treatment solution was used for pretreating the sample, a next operation can be proceeded without incubation.

### Test Example 2: Comparison of Testing Results Between Healthy People Samples and ILD Patient Samples

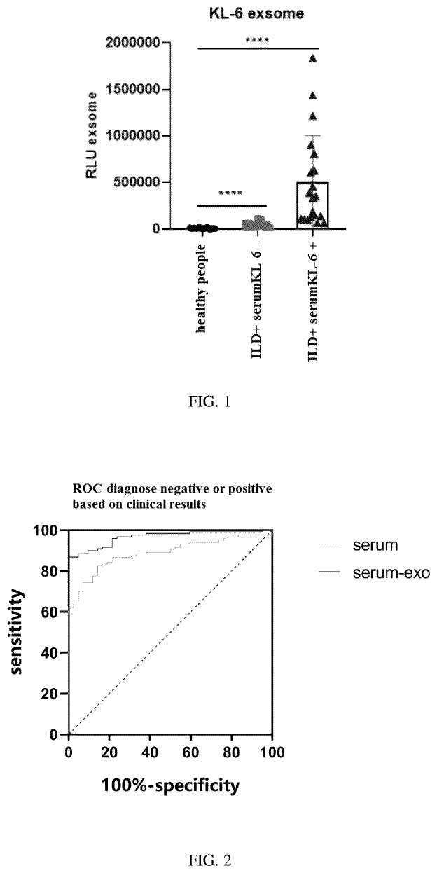

20 samples from ILD-positive patients with positive results from a commercial serum KL-6 detection reagent, 20 samples from ILD-positive patients with negative results from a commercial serum KL-6 detection reagent, and 20 serum samples from healthy people were taken, the method provided in Example 1 was used to detect the content of KL-6 protein on exosomes in the above samples. ILD negative and positive were determined based on high-resolution CT (HRCT) and other clinical outcomes. Test results are shown in Table 2 and FIG. 1.

TABLE 2
Comparison of testing results between healthy people samples and ILD patient samples
Detection luminescence value of KL-6 on exosomes

| Samples of serum KL-6 positive | | Samples of serum KL-6 negative | | | |
|---|---|---|---|---|---|
| and ILD positive | | and ILD positive | | Healthy people samples | |
| | Luminescence | | Luminescence | | Luminescence |
| Number | value | Number | value | Number | value |
| 1 | 1837726 | 21 | 57064 | 41 | 13667 |
| 2 | 101267 | 22 | 18999 | 42 | 10247 |
| 3 | 131003 | 23 | 54293 | 43 | 21592 |
| 4 | 69284 | 24 | 47133 | 44 | 6931 |
| 5 | 609573 | 25 | 16868 | 45 | 9637 |
| 6 | 457573 | 26 | 31906 | 46 | 9764 |
| 7 | 151717 | 27 | 63903 | 47 | 7096 |
| 8 | 108977 | 28 | 47839 | 48 | 7959 |
| 9 | 1216805 | 29 | 20346 | 49 | 4728 |
| 10 | 185600 | 30 | 21576 | 50 | 5632 |
| 11 | 810651 | 31 | 29553 | 51 | 5218 |
| 12 | 350942 | 32 | 42267 | 52 | 6825 |
| 13 | 138591 | 33 | 25636 | 53 | 6223 |
| 14 | 333768 | 34 | 49526 | 54 | 7305 |
| 15 | 906016 | 35 | 64593 | 55 | 4403 |
| 16 | 1437878 | 36 | 18711 | 56 | 15005 |
| 17 | 97948 | 37 | 99682 | 57 | 5049 |
| 18 | 65627 | 38 | 115801 | 58 | 5733 |
| 19 | 627338 | 39 | 23884 | 59 | 5851 |
| 20 | 390119 | 40 | 14409 | 60 | 23621 |

Analysis of data results: Referring to Table 2 and FIG. 1, the detection luminescence value of KL-6 protein on exosomes of the ILD-positive patients with both positive results and negative results from the commercial serum KL-6 detection reagent is significantly higher than the detection luminescence value of KL-6 protein on the exosomes of the healthy people samples. Very significant differences (P<0.0001) were shown between the patient samples with ILD positive and serum KL-6 positive and the healthy people samples, and between the patient samples with ILD positive and serum KL-6 negative and the healthy human samples.

The results show that for ILD positive patients which cannot be effectively detected by the detection of KL-6 in serum, the methods for detecting KL-6 protein on exosomes can identify most of them, and compared with the detection of KL-6 in serum, the detection of KL-6 on exosomes for interstitial pneumonia has obviously higher sensitivity and specificity.

### Test Example 3: Comparison of Clinical Coincidence Rate of Detection of KL-6 in Serum and KL-6 on Exosomes

Serum samples and exosome samples extracted from serum of ILD-positive patients and healthy people (60 cases in total) were simultaneously detected by using the method provided in Example 1, negative and positive samples were defined based on clinical diagnosis results of ILD, and ROC curves for detection results of KL-6 in serum and KL-6 on exosomes were plotted, respectively. Cutoff values were respectively determined based on a maximum value of Youden index (sensitivity+specificity−1). ILD negative and positive were determined based on high-resolution CT (HRCT) and other clinical outcomes. Test results are shown in FIG. 2.

Analysis of data results: According to the results shown in FIG. 2, the detection results of AUC value of KL-6 on exosomes is higher than that of KL-6 in serum, which indicates that the detection of KL-6 on exosomes has higher clinical coincidence rate in the ILD diagnosis process compared with the detection of KL-6 in serum. The difference in AUC between the detection of KL-6 on exosomes and the detection of KL-6 in serum is mainly reflected in specificity. The detection of KL-6 on exosomes can reduce the influence of the non-exosomal KL-6 proteins and other serum interferents in the sample, and has higher specificity (lower cutoff) compared to the detection of KL-6 in serum.

### Test Example 4: Combination of KL-6 and CA125 on Exosomes for Diagnosis of Interstitial Lung Diseases

Based on the ROC curve for detection results of KL-6 on exosomes from Test Example 3, the cutoff of a detection kit for KL-6 on exosomes was determined initially. Using the method provided in Example 9, a detection kit for CA125 on exosome was prepared, and the samples from Test Example 2 were tested to determine the cutoff initially. Based on the cutoff, the positivity or negativity of detection results of CA125 and KL-6 on exosomes of ILD patient samples was determined. Subsequently, a correlation of negative or positive of detection results of KL-6 and CA125 on exosomes in Test Example 2 with clinical symptoms of ILD patients was evaluated. Results are shown in Table 3.

TABLE 3
Results of a combination of KL-6 and CA125 on exosomes
for diagnosis of interstitial lung diseases

| | exo-KL-6 | exo-CA125 | |
|---|---|---|---|
| | (Luminescence | (Luminescence | IPF or severe |
| Number | value) | value) | pneumonia |
| 1 | 1837726 | 36099 | IPF |
| 2 | 101267 | 11638 | IPF |
| 3 | 131003 | 13768 | IPF |
| 4 | 69284 | 9166 | No lung disease |
| 5 | 609573 | 10697 | IPF |
| 6 | 457573 | 27179 | IPF |
| 7 | 151717 | 44568 | Severe |
| | | | pneumonia |
| 8 | 108977 | 8338 | No lung disease |
| 9 | 1216805 | 33024 | IPF |
| 10 | 185600 | 14686 | IPF |
| 11 | 810651 | 9907 | No lung disease |
| 12 | 350942 | 14694 | IPF |
| 13 | 138591 | 8074 | No lung disease |
| 14 | 333768 | 10184 | No lung disease |
| 15 | 906016 | 32414 | Severe |
| | | | pneumonia |
| 16 | 1437878 | 10474 | No lung disease |
| 17 | 97948 | 7954 | No lung disease |
| 18 | 65627 | 9384 | IPF |
| 19 | 627338 | 21404 | Severe |
| | | | pneumonia |
| 20 | 390119 | 21794 | IPF |
| 21 | 57064 | 20635 | Severe |
| | | | pneumonia |
| 22 | 18999 | 6406 | No lung disease |
| 23 | 54293 | 6394 | No lung disease |
| 24 | 47133 | 17065 | Respiratory |
| | | | failure |
| 25 | 16868 | 7896 | No lung disease |
| 26 | 31906 | 10214 | No lung disease |
| 27 | 63903 | 9455 | No lung disease |
| 28 | 47839 | 9798 | No lung disease |
| 29 | 20346 | 7824 | No lung disease |
| 30 | 21576 | 11415 | No lung disease |
| 31 | 29553 | 13216 | No lung disease |
| 32 | 42267 | 9904 | No lung disease |
| 33 | 25636 | 9385 | No lung disease |
| 34 | 49526 | 9784 | No lung disease |
| 35 | 64593 | 9938 | No lung disease |
| 36 | 18711 | 8548 | No lung disease |
| 37 | 99682 | 21950 | IPF |
| 38 | 115801 | 8398 | Severe |
| | | | pneumonia |
| 39 | 23884 | 10076 | No lung disease |
| 40 | 14409 | 13658 | No lung disease |

Result analysis: Based on the ROC curve, the cutoff value for CA125 on exosomes is initially determined to be 11053, and the cutoff value for KL-6 on exosomes is initially determined to be 39102. There were 14 samples positive for both KL-6 and CA125 on exosomes, accounting for 82% (14/17) of all cases identified as either Idiopathic Pulmonary Fibrosis (IPF) or severe pneumonia. There were 16 samples positive for KL-6 on exosomes but negative for CA125 on exosomes, with only 3 cases among these diagnosed with IPF or severe pneumonia, accounting for 18.7% (3/16) of all cases identified as non-IPF or non-severe pneumonia.

The results show that a combined diagnostic of KL-6 and CA125 on exosomes can effectively identify most of IPF or severe pneumonia cases. A combined detection of KL-6 and CA125 on exosomes may have significant clinical value in distinguishing interstitial pneumonia and assessing a severity of interstitial pneumonia.

It should be noted that the above-described Examples are only for explaining the present application and do not constitute any limitation of the present application. The application has been described with reference to exemplary Examples, but it is understood that the words used are descriptive and explanatory, not restrictive. Modifications can be made to the present application within a scope of the claims as stipulated, and revisions can be made to the present invention without departing from a scope and spirit of the present application. Although the present application is described herein with reference to particular means, materials and Examples, the present application is not intended to be limited to the particular Examples disclosed, rather, the application extends to all functionally equivalent methods and uses.

What is claimed is:

1. Use of KL-6 protein on exosomes in preparing a kit for diagnosing a lung disease.

2. Use of a combination of KL-6 and CA125 on exosomes in preparing a kit for diagnosing a lung disease.

3. The use according to claim 1, wherein the kit comprises a sample treatment solution; and the sample treatment solution is a lysis type sample treatment solution or a non-lysis type sample treatment solution.

4. The use according to claim 3, wherein the lysis type sample treatment solution comprises the following raw materials in percentage by weight: disodium hydrogen phosphate dodecahydrate 0.5-2%, sodium dihydrogen phosphate dihydrate 0.5-2%, NaCl 0.5-3.0%, Bovine Serum Albumin (BSA) 0.5-3%, polyethylene glycol octylphenyl ether (Triton X-100) 0.05-1%, anionic surfactant 0.1-2%, zwitterionic surfactant 0.1-5%, polysorbate 20 (Tween-20) 0.1-2%, ethylenediaminetetraacetic acid (EDTA)-2Na 0.02-0.1%, sodium azide 0.05-0.2%, and water in balance.

5. The use according to claim 4, wherein the anionic surfactant is at least one selected from a group consisting of: sodium dodecyl sulfate, sodium lauroyl sarcosinate, and lithium dodecyl sulfate; and the zwitterionic surfactant is at least one selected from a group consisting of: 3-[(3-cholamidopropyl)dimethylammonio]-1-propanesulfonate (CHAPS), sodium lauroamphoacetate, sodium cocoamphoacetate, 3-[(3-cholamidopropyl)dimethylammonio]-2-hydroxy-1-propanesulfonate (CHAPSO), 3-(N,N-Dimethylmyristylammonio) propanesulfonate (SB3-14), 3-(N,N-Dimethyllaurylammonio) propanesulfonate (SB3-12), 3-(Decyldimethylammonio)-propane-sulfonate inner salt (SB3-10), and 3-(N,N-Dimethylpalmitylammonio) propanesulfonate (SB3-8).

6. The use according to claim 3, wherein the non-lysis type sample treatment solution comprises the following raw materials in percentage by weight: disodium hydrogen phosphate dodecahydrate 0.5-2%, sodium dihydrogen phosphate dihydrate 0.5-2%, NaCl 0.5-3%, Bovine Serum Albumin (BSA) 0.5-3%, zwitterionic surfactant 0.2-2%, polysorbate 20 (Tween-20) 0.1-2%, ethylenediaminetetraacetic acid (EDTA)-2Na 0.02-0.1%, sodium azide 0.05-0.2%, and water in balance.

7. The use according to claim 6, wherein the zwitterionic surfactant is at least one selected from a group consisting of: 3-(N,N-Dimethylmyristylammonio) propanesulfonate (SB3-14), sodium lauroamphoacetate, sodium cocoamphoacetate, 3-(N,N-Dimethyllaurylammonio) propanesulfonate (SB3-12), 3-(Decyldimethylammonio)-propane-sulfonate inner salt (SB3-10), and 3-(N,N-Dimethylpalmitylammonio) propanesulfonate (SB3-8).

8. A kit for diagnosing a lung disease, comprising the sample treatment solution according to claim 3, a magnetic bead coated with a capture antibody, a detection antibody labeled with a luminescent marker, a luminescent substrate, and a protein standard.

9. The kit for diagnosing a lung disease according to claim 8, wherein the capture antibody is a KL-6 capture antibody, and the detection antibody is a KL-6 detection antibody; or the capture antibody is a CA125 capture antibody, and the detection antibody is a CA125 detection antibody.

10. A method for detecting protein content on exosomes using the kit according to claim 8, comprising the following steps:
before detecting an exosome sample to be detected, performing a pretreatment on the exosome sample to be detected by mixing the exosome sample to be detected with the sample treatment solution to obtain a pretreated exosome sample to be detected;
wherein, a volume ratio of the exosome sample to be detected to the sample treatment solution is 1:(2-30);
a reaction time of the pretreatment is 1-120 min; and
a reaction temperature of the pretreatment is 25-42° C.
